(19) United States
(12) Patent Application Publication
(10) Pub. No.: US 2025/0236216 A1
(43) Pub. Date: Jul. 24, 2025

(54) ELECTRONIC DEVICE AND CONTROL METHOD THEREOF

(71) Applicants: HYUNDAI MOTOR COMPANY, Seoul (KR); Kia Corporation, Seoul (KR)

(72) Inventors: Ki Chang KIM, Hwaseong-si (KR); Dong Chul PARK, Hwaseong-si (KR)

(21) Appl. No.: 18/941,929

(22) Filed: Nov. 8, 2024

(30) Foreign Application Priority Data
Jan. 23, 2024 (KR) 10-2024-0009994

(51) Int. Cl. B60N 2/02; A61B 5/00; B60N 2/00; G07C 5/02

(52) U.S. Cl. CPC B60N 2/0273; A61B 5/4561; A61B 5/486; A61B 5/6893; B60N 2/0024; B60N 2/026; B60N 2/0268; G07C 5/02; A61B 2562/0247

(57) ABSTRACT

An electronic device performs a control method. The electronic device obtains information of at least one of a sitting posture of a user or driving information of a vehicle, or any combination thereof using sensors, determines whether motion sickness is caused in the user based on the information, and provides a motion sickness reduction solution based on determining that the motion sickness is caused in the user.

### CROSS-REFERENCE TO RELATED APPLICATION

This application claims the benefit of priority to Korean Patent Application No. 10-2024-0009994, filed in the Korean Intellectual Property Office, on Jan. 23, 2024, the entire contents of which are incorporated herein by reference.

### TECHNICAL FIELD

The present disclosure relates to an electronic device loaded into a vehicle and a control method thereof.

### BACKGROUND

With the development of autonomous driving technology, passengers in a vehicle during autonomous driving have enjoyed various pieces of contents, such as a virtual reality (VR) game. In some cases, when the passenger uses VR content while a vehicle is traveling, motion sickness with symptoms, such as dizziness and/or vomiting, that is, cybersickness may be caused by vibration due to the movement of the vehicle and/or a sitting posture of the passenger. The passenger may experience inconvenience if using VR content in the vehicle while the vehicle is traveling.

### SUMMARY

The present disclosure describes an electronic device for determining whether there is a possibility that cybersickness may occur in a user who uses virtual reality (VR) content based on a sitting posture of the user and/or a driving information of a vehicle and a control method thereof.

The present disclosure further describes an electronic device for correcting a sitting posture of a user to reduce cybersickness and controlling a vehicle seat to avoid resonance between an external excitation frequency and the natural frequency of the human body and a control method thereof.

According to one aspect of the subject matter described in this application, an electronic device includes one or more sensors, and a processor. The processor is configured to obtain, from the one or more sensors, motion information including at least one of a sitting posture of a user of a vehicle or driving information of the vehicle, or any combination thereof, determine whether the user experiences motion sickness based on the motion information, and provide a motion sickness reduction solution based on determining that the user experiences the motion sickness.

Implementations according to this aspect can include one or more of the following features. For example, the one or more sensors may include a pressure sensor, where the processor is configured to measure a body pressure distribution on a seat back and a seat cushion of the vehicle using the pressure sensor. In some examples, the processor is configured to determine whether load distribution of the sitting posture is eccentric based on the body pressure distribution, and determine that the user experiences the motion sickness based on determining that the load distribution of the sitting posture is eccentric.

In some implementations, the one or sensors include a vehicle sensor, where the processor is configured to determine a behavior of the vehicle based on the driving information obtained by the vehicle sensor, determine whether an excitation frequency according to the behavior of the vehicle resonates with a natural frequency of a human body, and determine that the user experiences the motion sickness based on determining that the excitation frequency according to the behavior of the vehicle resonates with the natural frequency of the human body. In some examples, the processor is configured to determine whether the behavior of the vehicle corresponds to a sudden cornering, sudden braking, or sudden acceleration.

In some implementations, the processor is configured to determine whether the motion sickness is caused by the sitting posture of the user, and correct the sitting posture based on determining that the motion sickness is caused by the sitting posture. In some examples, the processor is configured to adjust an amount of air in at least one of (i) a first air tube included in a neck pillow of a vehicle seat or (ii) a second air tube included in a seat cushion of the vehicle seat, or any combination thereof, to thereby adjust load distribution of the sitting posture to be even. In some examples, the processor is configured to output a notification suggesting correction of the sitting posture.

In some implementations, the processor is configured to determine whether the motion sickness is caused by a resonance with a natural frequency of a human body, and perform a resonance avoidance control based on determining that the motion sickness is caused by the resonance with the natural frequency of the human body. In some examples, the processor is configured to expand at least one of (i) a first air tube included in a neck pillow of a vehicle seat or (ii) a second air tube included in a seat cushion of the vehicle seat, or any combination thereof, to thereby change the natural frequency of the human body.

According to another aspect, a control method of an electronic device includes obtaining, from one or more sensors, motion information including at least one of a sitting posture of a user of a vehicle or driving information of the vehicle, or any combination thereof, determining whether the user experiences motion sickness based on the motion information, and providing a motion sickness reduction solution based on determining that the user experiences the motion sickness.

Implementations according to this aspect can include one or more of the following features. For example, the one or more sensors include a pressor sensor and a vehicle sensor, where obtaining the motion information includes measuring body pressure distribution on a seat back and a seat cushion of a vehicle seat of the vehicle, and obtaining the driving information of the vehicle using the vehicle sensor.

In some implementations, determining whether the user experiences the motion sickness includes determining whether load distribution of the sitting posture is eccentric based on the body pressure distribution, and determining that the user experiences the motion sickness based on determining that the load distribution of the sitting posture is eccentric.

In some examples, determining whether the user experiences the motion sickness includes determining a behavior of the vehicle based on the driving information of the vehicle, determining whether an excitation frequency according to the behavior of the vehicle resonates with a natural frequency of a human body, and determining that the user experiences the motion sickness based on determining that the excitation frequency according to the behavior of the vehicle resonates with the natural frequency of the human body. In some examples, determining the behavior of the vehicle includes determining whether the behavior of the vehicle is a sudden cornering, sudden braking, or sudden acceleration.

In some implementations, providing the motion sickness reduction solution includes determining whether the motion sickness is caused by the sitting posture, and correcting the sitting posture based on determining that the motion sickness is caused by the sitting posture. For example, correcting the sitting posture includes adjusting an amount of air in at least one of (i) a first air tube included in a neck pillow of the vehicle seat or (ii) a second air tube included in the seat cushion, or any combination thereof, to thereby adjust load distribution in the sitting posture to be even. In some examples, correcting the sitting posture includes outputting a notification that suggests correcting the sitting posture.

In some implementations, providing the motion sickness reduction solution includes determining whether the motion sickness is caused by a resonance with a natural frequency of a human body, and performing a resonance avoidance control based on determining that the motion sickness is caused by the resonance with the natural frequency of the human body. For example, performing the resonance avoidance control includes expanding at least one of (i) a first air tube included in a neck pillow of the vehicle seat or (ii) a second air tube included in the seat cushion, or any combination thereof, to thereby change the natural frequency of the human body.

### BRIEF DESCRIPTION OF THE DRAWINGS

The above and other objects, features and advantages of the present disclosure will be more apparent from the following detailed description taken in conjunction with the accompanying drawings.

FIG. 1 is a block diagram illustrating an example of an electronic device.

FIG. 2 is a drawing illustrating an example of an air tube mounting structure in a vehicle seat.

FIGS. 3A and 3B are drawings illustrating an example of controlling a vehicle seat.

FIG. 4 is a flowchart illustrating an example of a control method of an electronic device.

FIG. 5 is a drawing illustrating an example of a control method of an electronic device.

### DETAILED DESCRIPTION

Hereinafter, some implementations of the present disclosure will be described in detail with reference to the example drawings. In adding the reference numerals to the components of each drawing, it should be noted that the identical component is designated by the identical numerals even when they are displayed on other drawings. In addition, a detailed description of well-known features or functions will be ruled out in order not to unnecessarily obscure the gist of the present disclosure.

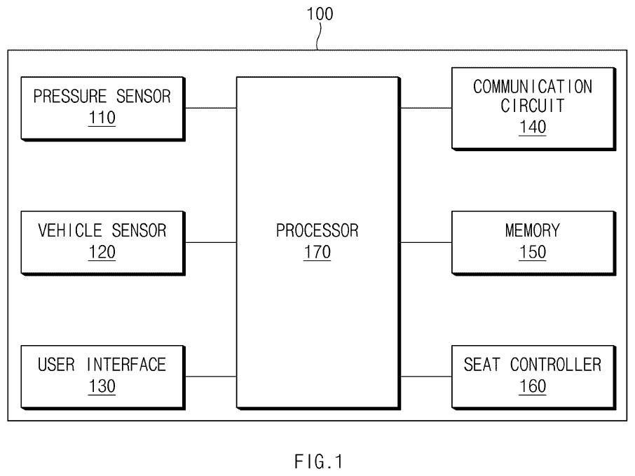

FIG. 1 is a block diagram illustrating an example configuration of an electronic device.

In some implementations, an electronic device 100 may be loaded into a vehicle capable of performing autonomous driving. Referring to FIG. 1, the electronic device 100 may include a pressure sensor 110, a vehicle sensor 120, a user interface 130, a communication circuit 140, a memory 150, a seat controller 160, and a processor 170.

In some examples, the pressure sensor 110 may be mounted on each of a seat back and a seat cushion of a vehicle seat. The pressure sensor 110 may measure body pressure applied to the seat back and body pressure applied to the seat cushion.

In some implementations, the vehicle sensor 120 may detect vehicle information, such as a steering angle, a vehicle speed, acceleration, seat information, a motor revolution per minute (RPM), an accelerator pedal opening amount, a brake pedal opening amount, and/or a throttle opening amount, using at least one sensor and/or at least one electronic control unit (ECU), which are/is mounted on the vehicle. The at least one sensor may include an accelerator position sensor (APS), a throttle position sensor, a global positioning system (GPS) sensor, a wheel speed sensor, an image sensor (or a camera), an advanced driver assistance system (ADAS) sensor, a 3-axis accelerometer, a gyro sensor, an inertial measurement unit (IMU), and/or the like. The at least one ECU may be a motor control unit (MCU), a vehicle control unit (VCU), and/or the like.

The user interface 130 may support an interaction between the electronic device 100 and a user. The user interface 130 may include an input device (e.g., a keyboard, a touch pad, a microphone, a touch screen, and/or the like) for generating data according to manipulation of the user, an output device (e.g., a display, a head-up display (HUD), a speaker, a tactile signal output device, and/or the like) for outputting information according to an operation of a vehicle system, and/or the like.

The communication circuit 140 may support to perform wired communication or wireless communication between the electronic device 100 and an external electronic device (e.g., a VR device, a wearable device, an ECU, or the like). The communication circuit 140 may include an electric circuit, a wired communication circuit (e.g., a LAN communication circuit and/or a power line communication circuit), a wireless communication circuit (e.g., a mobile communication circuit, a short range wireless communication circuit, a global navigation satellite system (GNSS) communication circuit), and/or the like.

The memory 150 may store VR content. The memory 150 may store an algorithm for determining whether motion sickness is caused, a control algorithm for reducing motion sickness, and/or the like. Furthermore, the memory 150 may store input data and/or output data of the processor 170. The memory 150 may store setting information preset by a designer.

The memory 150 may be a non-transitory storage medium which stores instructions executed by the processor 170. The memory 150 may include at least one of storage media such as a random access memory (RAM), a static RAM (SRAM), a read only memory (ROM), a programmable ROM (PROM), an electrically erasable and programmable ROM (EEPROM), an erasable and programmable ROM (EPROM), a hard disk drive (HDD), a solid state disk (SSD), an embedded multimedia card (eMMC), universal flash storage (UFS), or web storage.

The seat controller 160 may control expansion or contraction of air tube(s) mounted in a neck pillow mounted on the vehicle seat and/or the seat cushion. The seat controller 160 may adjust an amount of air in the air tube in the neck pillow depending on a control command transmitted from the processor 170. Furthermore, the seat controller 160 may adjust an amount of air in the air tube in the seat cushion depending on a control command transmitted from the processor 170.

The processor 170 may be connected with the respective components 110 to 160 to control operations of the respective components 110 to 160. The processor 170 may include at least one of an electric circuit, an application specific integrated circuit (ASIC), a digital signal processor (DSP), programmable logic devices (PLD), field programmable gate arrays (FPGAS), a central processing unit (CPU), microcontrollers, or microprocessors, or any combination thereof.

The processor 170 may receive data for instructing to execute VR content (e.g., a VR game or the like) from the user interface 130, while the vehicle is performing autonomous driving (or is traveling). The processor 170 may execute the VR content based on the received data. The processor 170 may output a virtual image and a virtual sound of the executed VR content to the user interface 130. The user interface 130 may output the virtual image on the display and may output the virtual image through the speaker.

While executing the VR content, the processor 170 may obtain a sitting posture of the user, driving information of the vehicle, and/or the like using the pressure sensor 110 and the vehicle sensor 120. The processor 170 may determine whether there is a possibility that motion sickness (or cybersickness) may occur in the user based on at least one of the sitting posture of the user or the driving information of the vehicle, or any combination thereof. Furthermore, the processor 170 may determine a motion sickness factor based on at least one of the sitting posture of the user or the driving information of the vehicle, or any combination thereof. In other words, the processor 170 may determine whether the motion sickness occurring is motion sickness due to the sitting posture or motion sickness due to resonance between an external excitation frequency and the natural frequency of the human body.

The processor 170 may measure body pressure distribution (or surface pressure distribution), maximum body pressure, and/or the like on the seat back and the seat cushion using the pressure sensor 110. The processor 170 may determine whether load distribution is equal distribution or eccentric distribution based on the measured body pressure distribution and/or the measured maximum body pressure. If it is determined that the load distribution is the equal distribution, the processor 170 may determine the sitting posture of the user does not cause motion sickness. In some examples, if it is determined that the load distribution is the eccentric distribution, the processor 170 may determine the sitting posture of the user causes the motion sickness. Furthermore, if it is determined that the load distribution is the eccentric distribution, the processor 170 may determine the cause of motion sickness (or a motion sickness factor) as the sitting posture (or postural instability) of the user.

As an example, if weight distribution between the back and the thighs of the user is 5:5, the processor 170 may determine the load distribution of the sitting posture as the equal distribution. As another example, if the weight distribution between the back and the thighs of the user is 3:7, the processor 170 may determine the load distribution of the sitting posture as the eccentric distribution.

If it is determined that the sitting posture of the user causes the motion sickness, the processor 170 may adjust an amount of air in the air tube(s) included in the neck pillow and/or the seat cushion of the vehicle seat. The processor 170 may increase the amount of air in the air tube to support the neck and/or the thighs of the user, thus correcting the sitting posture of the user. If it is determined that the sitting posture of the user causes the motion sickness, the processor 170 may output information (e.g., visual information, audible information, tactile information, and/or the like) indicating that there is a need to correct the sitting posture of the user on the user interface 130.

Furthermore, if it is determined that the sitting posture of the user causes the motion sickness, the processor 170 may inject air into the air tube(s) included in the neck pillow and/or the seat cushion of the vehicle seat to expand the air tube. The processor 170 may help to correct the sitting posture of the user through the expansion of the air tube(s) included in the neck pillow and/or the seat cushion. As the fatigue of the user is reduced by the correction of the sitting posture, the motion sickness may be alleviated.

The processor 170 may determine whether there is a possibility that motion sickness may occur in the user based on the driving information of the vehicle, which is obtained by the vehicle sensor 120. The processor 170 may determine a behavior (e.g., sudden cornering, sudden braking, sudden acceleration, or the like) based on vehicle information.

The processor 170 may determine whether the vehicle is in a sudden cornering situation based on the driving information. For example, if the steering angle of the vehicle changes to a predetermined reference steering angle or more or if the rotational speed of the vehicle is greater than or equal to a predetermined reference speed, the processor 170 may determine that the vehicle is in the sudden cornering situation. Low-frequency vibration of 1 Hz or less may occur upon sudden cornering. Motion sickness may occur due to heavy vibration (i.e., resonance) by an overlap between an excitation frequency (e.g., an external excitation frequency) of the vibration and the natural frequency of the human body.

The processor 170 may determine whether the vehicle is a sudden braking situation or a sudden acceleration situation based on the vehicle information. As an example, if the acceleration is greater than predetermined reference acceleration, the processor 170 may determine that the vehicle is in the sudden acceleration situation. As another example, if the deceleration is less than or equal to predetermined reference deceleration, the processor 170 may determine that the vehicle is in the sudden braking situation. Upon sudden acceleration or sudden braking, excitation frequency resonates with a camouflage natural frequency of 4 to 8 Hz to cause motion sickness.

If it is determined that the vehicle is in the sudden cornering situation, the sudden braking situation, or the sudden acceleration situation, the processor 170 may inject air into the air tube of the vehicle seat to expand the air tube for resonance avoidance. If the excitation frequency according to the behavior of the vehicle overlaps (or resonates) with the natural frequency of the human body, the processor 170 may determine that motion sickness occurs due to resonance. The processor 170 may expand the air tube included in the vehicle seat to perform resonance avoidance.

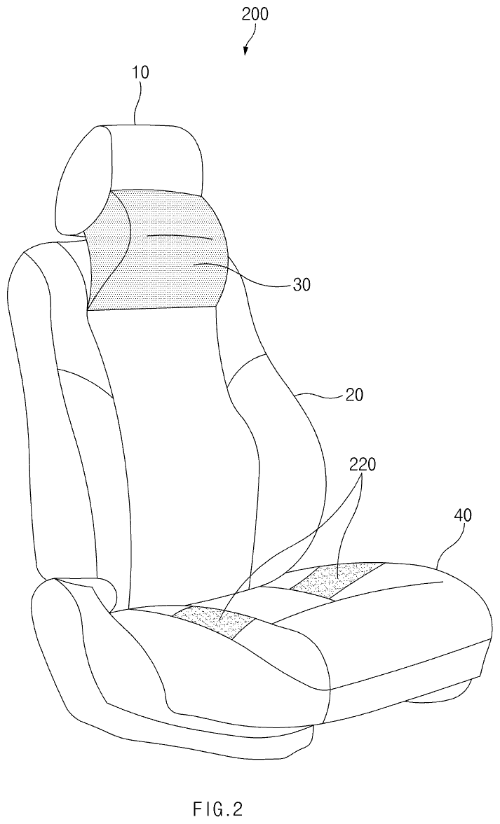

FIG. 2 is a drawing for describing an air tube mounting structure in a vehicle seat.

A vehicle seat 200 may include a first air tube 210 and second air tubes 220, which are expanded or contracted by the amount of injected air.

The first air tube 210 may be embedded in a neck pillow 30 detachably mounted on a boundary between a headrest 10 and a seat back 20 If air is injected into the first air tube 210 and the first air tube 210 is expanded, the neck pillow 30 may support the neck of a user sitting on the vehicle seat 200 to correct a sitting posture of the user, thus avoiding resonance.

The second air tubes 220 may be mounted to be symmetrical on both sides of a seat cushion 40. If air is injected into the second air tubes 220 and the second air tubes 220 are expanded, the seat cushion 40 may support the thighs of the user sitting on the vehicle seat 200 to correct a sitting posture of the user, thus avoiding resonance.

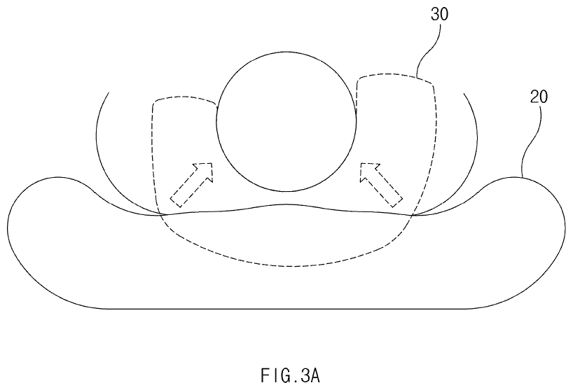

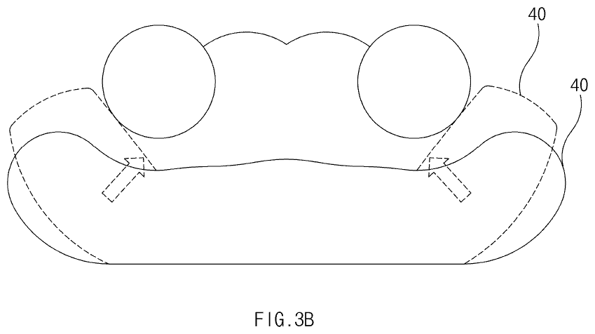

FIGS. 3A and 3B are drawings illustrating an example of controlling a vehicle seat associated with the present disclosure.

Referring to FIG. 3A, if it is determined that motion sickness occurs in a user sitting on a vehicle seat 200, a processor 170 may instruct a seat controller 160 to inject air into a first air tube 210 in a neck pillow 30. The seat controller 160 may inject air into the first air tube 210 to expand the first air tube 210. As the first air tube 210 is expanded, the neck pillow 30 may support the neck of the user sitting on the vehicle seat 200 to help to correct a sitting posture of the user, thus avoiding resonance.

Referring to FIG. 3B, if it is determined that there is a possibility that motion sickness may occur in the user sitting on the vehicle seat 200, the processor 170 may transmit a control command to instruct to inject air into second air tubes 220 included in a seat cushion 40 to the seat controller 160. The seat controller 160 may adjust an amount of air in the second air tubes 220 depending on the control command of the processor 170 to expand the second air tubes 220. As the second air tubes 220 are expanded, the seat cushion 40 may support the thighs of the user sitting on the vehicle seat 200 to help to correct a sitting posture of the user, thus avoiding that an excitation frequency according to the behavior of the vehicle resonates with the natural frequency of the human body.

In some implementations, if the first air tube 210 of the neck pillow 30 and the second air tubes 220 of the seat cushion 40 are expanded, the human body's natural vibration frequency may fluctuate due to an increase in weight due to the expansion. This may lead to posture correction and resonance avoidance.

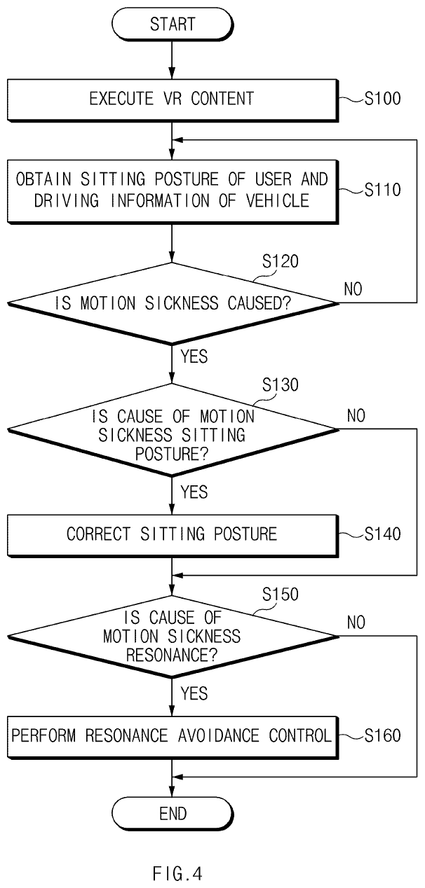

FIG. 4 is a flowchart illustrating an example of a control method of an electronic device.

In S100, a processor 170 may execute VR content depending to a request received from the outside while driving. The processor 170 may receive data for instructing to execute VR content (e.g., a VR game or the like) from a user interface 130, while a vehicle is traveling. The processor 170 may execute the VR content based on the received data.

In S110, the processor 170 may obtain a sitting posture of a user and driving information of the vehicle using sensors mounted on the vehicle. The processor 170 may measure body pressure (or surface pressure) distribution of a seat back 20 and a seat cushion 40 of the vehicle seat 200 using a pressure sensor 110. The processor 170 may measure the driving information of the vehicle using a vehicle sensor 120.

In S120, the processor 170 may determine motion sickness is caused based on at least one of the sitting posture of the user or the driving information of the vehicle, or any combination thereof. The processor 170 may determine whether there is a possibility that motion sickness may occur in the user based on at least one of the sitting posture of the user or the driving information of the vehicle, or any combination thereof.

As an example, the processor 170 may determine whether there is a possibility that the motion sickness may occur in the user due to the sitting posture of the user. The processor 170 may determine whether load distribution on the vehicle seat 200 is equal distribution or eccentric distribution based on measured body pressure distribution. If it is determined that the load distribution on the vehicle seat 200 is the eccentric distribution, the processor 170 may determine the sitting posture of the user as a posture causing the motion sickness. In some examples, if it is determined that the load distribution on the vehicle seat 200 is the equal distribution, the processor 170 may determine the sitting posture of the user as a posture which does not cause the motion sickness.

For example, referring to Table 1 below, if the load distribution in the sitting posture is 3:7 and is eccentric (a second sitting posture) as the result of analyzing body pressure distribution on the seat back 20 and the seat cushion 40 of the vehicle seat 200, as the weight of the user is concentrated on the lower part (e.g., thighs), cybersickness making the user feel physically tired and sick to his or her stomach may occur. Because maximum body pressure is higher and the sensitivity of thighs is relatively higher than the back if the weight of the user is concentrated on the lower part than if the load distribution of the sitting posture is 5:5 and is even (a first sitting posture), the user exerts force on the lower part of the body to balance the body, fatigue may increase. This may cause cybersickness.

TABLE 1

| | Maximum | | | |
|---|---|---|---|---|
| | body | Distribution | Sensitivity | Sensitivity |
| Classification | pressure | Area | of back | of thighs |
| First sitting | 1.8 N/cm² | 2939 cm² | 5~25 | 5~20 |
| posture | | | | |
| Second sitting | 2.5 N/cm² | 2376 cm² | 6~32 | 14~40 |
| posture | | | | |

In some implementations, the processor 170 may determine a behavior of the vehicle based on the driving information of the vehicle. The processor 170 may determine whether there is a possibility that motion sickness may occur in the user based on the behavior of the vehicle. The processor 170 may determine whether the vehicle is in a sudden cornering situation, a sudden acceleration situation, or a sudden braking situation based on the driving information of the vehicle.

If the vehicle is in the sudden cornering situation, the sudden acceleration situation, or the sudden braking situation, the processor 170 may determine that there is the possibility that the motion sickness may occur in the user. For example, when the vehicle performs sudden cornering, a left and right excitation frequency of 1 Hz or less (i.e., low frequency motion) may occur at a seat waist portion. At this time, if the user exerts excessive force on his or her thighs to maintain the posture, this may cause motion sickness as well as causing physical fatigue. Furthermore, when the vehicle performs sudden acceleration or sudden braking, a front and rear excitation frequency of 4 Hz to 8 Hz may occur at a seat back portion. Motion sickness may occur due to heavy vibration by resonance between the excitation frequency and a camouflage natural frequency (i.e., an overlap between the excitation frequency and the camouflage natural frequency).

If it is determined that there is the possibility that the motion sickness may occur in the user, in S130, the processor 170 may determine the cause of the motion sickness is a sitting posture.

If it is determined that the cause of the motion sickness is the sitting posture, in S140, the processor 170 may output posture correction information. The processor 170 may adjust an amount of air in a first air tube 210 included in a neck pillow 30 and/or second air tubes 220 included in the seat cushion 40 to correct the sitting posture of the user. If it is determined that the sitting posture of the user causes the motion sickness, the processor 170 may output a notification indicating that there is a need to correct the sitting posture in the form of at least one of visual information, audible information, or tactile information, or any combination thereof.

If it is determined that the cause of the motion sickness is not the sitting posture, in S150, the processor 170 may determine whether the cause of the motion sickness is resonance. In other words, the processor 170 may determine whether an external excitation frequency and the natural frequency of the human body overlap with each other based on the driving information of the vehicle. If it is determined that the vehicle is in the sudden cornering situation, the sudden braking situation, or the sudden acceleration situation, the processor 170 may determine that the cause of the motion sickness is the resonance.

If it is determined that the cause of the motion sickness is the resonance, in S160, the processor 170 may perform resonance avoidance control. The processor 170 may adjust an amount of air in the first air tube 210 and/or the second air tube 220 of the vehicle seat 200 for resonance avoidance, thus controlling the resonance avoidance. If the vehicle is in the sudden cornering situation, the sudden braking situation, or the sudden acceleration situation, the processor 170 may inject air into the first air tube 210 and/or the second air tubes 220 of the vehicle seat 200 to expand the first air tube 210 and/or the second air tubes 220 for resonance avoidance.

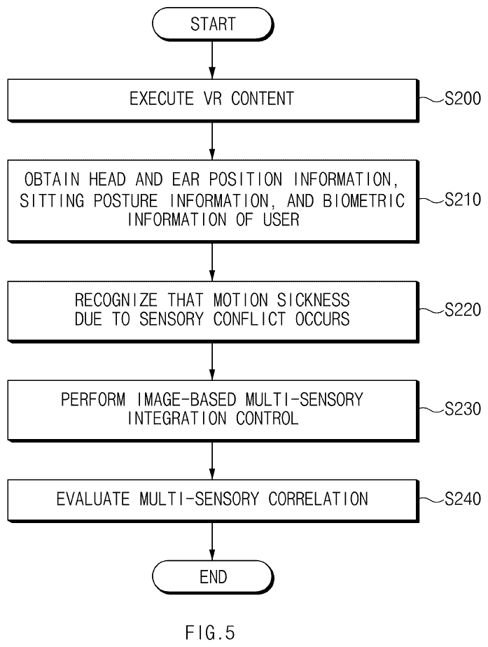

FIG. 5 is a drawing for describing an example of a control method of an electronic device.

In S200, a processor 170 of an electronic device 100 may execute VR content depending to a request received from the outside while driving. The processor 170 may receive data for instructing to execute VR content (e.g., a VR game or the like) from a user interface 130, while a vehicle is traveling. The processor 170 may execute the VR content based on the received data.

In S210, the processor 170 may obtain head and ear position information, sitting posture information, and biometric information of a user using sensors, when executing the VR content. The processor 170 may obtain the head and ear position information of the user using a head-related transfer function (HRTF). The processor 170 may measure body pressure (e.g., the back and the thighs) on a seat back and a seat cushion and may analyze the measured body pressure to determine a sitting posture. Furthermore, the processor 170 may recognize biometric information of the user, for example, heart rate variability and eye movement, using a camera and a wearable device.

In S220, the processor 170 may recognize that motion sickness due to a sensory conflict occurs in the user based on the head and ear position information, the sitting posture information, and the biometric information of the user. In detail, the processor 170 may determine a motion sickness stage of the user based on the head and ear position information, the sitting posture information, and the biometric information of the user. The motion sickness stage may be divided into a normal stage, a motion sickness boundary stage, a motion sickness stage due to postural instability, a motion sickness stage due to a sensory conflict, and the like.

If it is recognized that the motion sickness due to the sensory conflict occurs in the user, in S230, the processor 170 may perform multi-sensory integration control (or image-based multi-sensory integration control) based on a virtual image of the VR content. In other words, the processor 170 may control a sound, motion, vibration/haptics based on the virtual image of the VR content. The processor 170 may improve motion sickness due to postural instability by multiple cognitive characteristic immersion through the image-based multi-sensory integration control.

The processor 170 may convert an image characteristic into a sound factor based on a convolutional neural network (CNN) algorithm, may perform 3D sounding recording based on personalized physical characteristic logic, and may encode and decode a stereoscopic sound (or a 3D sound), thus playing an image-linked sound in an ultra-realistic way at ear positions of the user. The processor 170 may convert an image characteristic into a motion factor based on artificial neural network learning, may tune a 6-degrees of freedom motion seat based on an autonomous driving scenario, and may analyze personalization immersion deviation by multi-modal excitation, thus implementing image-linked motion scenario-based immersion. The processor 170 may convert an image characteristic into a vibration and haptic (vibration/haptic) factor based on a multi-displacement response measurement algorithm, may apply time delay prevention logic, and may tune intensity and a pattern of a vibration profile, thus implementing image-linked haptics on a VR goggle-based haptic controller.

In S240, the processor 170 may evaluate a multi-sensory correlation, while performing the image-based multi-sensory integration control. At this time, the processor 170 may evaluate the multi-sensory correlation for each test mode, such as a sound compared to an image, motion compared to the image, and vibration/haptics compared to the image. The processor 170 may determine biometric information of the user using sensing devices (e.g., a camera, a wearable device, and the like). The processor 170 may determine a state of the user based on the biometric signal detected by the sensing devices. For example, the processor 170 may analyze at least one of heart rate variability, eye movement, a brain wave pattern, or any combination thereof using the sensing devices. The processor 170 may determine the state of the user as good, borderline motion sickness, progressive motion sickness, severe motion sickness, or the like based on the analyzed result.

Thereafter, the processor 170 may output the result of evaluating the multi-sensory correlation and may train image-based multi-sensory integration control logic stored in a memory 150.

In some implementations, the electronic device may recognize whether there is a possibility that cybersickness may occur in the user who uses virtual reality (VR) content based on the sitting posture of the user and/or the driving information of the vehicle.

In some implementations, the electronic device may correct the sitting posture of the user in the situation in which there is the possibility that the cybersickness may occur in the user and may control a vehicle seat to avoid resonance between an external excitation frequency and the natural frequency of the human body, thus reducing (or alleviating) the cybersickness.

Hereinabove, although the present disclosure has been described with reference to example implementations and the accompanying drawings, the present disclosure is not limited thereto, but may be variously modified and altered by those skilled in the art to which the present disclosure pertains without departing from the spirit and scope of the present claims. Therefore, disclosure claimed in the following implementations of the present disclosure are not intended to limit the technical spirit of the present disclosure, but provided only for the illustrative purpose. The scope of the present disclosure should be construed on the basis of the accompanying claims, and all the technical ideas within the scope equivalent to the claims should be included in the scope of the present disclosure.

What is claimed is:

1. An electronic device, comprising:
one or more sensors; and
a processor configured to:
obtain, from the one or more sensors, motion information including at least one of a sitting posture of a user of a vehicle or driving information of the vehicle, or any combination thereof,
determine whether the user experiences motion sickness based on the motion information, and
provide a motion sickness reduction solution based on determining that the user experiences the motion sickness.

2. The electronic device of claim 1, wherein the one or more sensors include a pressure sensor, and
wherein the processor is configured to measure a body pressure distribution on a seat back and a seat cushion of the vehicle using the pressure sensor.

3. The electronic device of claim 2, wherein the processor is configured to:
determine whether load distribution of the sitting posture is eccentric based on the body pressure distribution; and
determine that the user experiences the motion sickness based on determining that the load distribution of the sitting posture is eccentric.

4. The electronic device of claim 1, wherein the one or sensors include a vehicle sensor, and
wherein the processor is configured to:
determine a behavior of the vehicle based on the driving information obtained by the vehicle sensor;
determine whether an excitation frequency according to the behavior of the vehicle resonates with a natural frequency of a human body; and
determine that the user experiences the motion sickness based on determining that the excitation frequency according to the behavior of the vehicle resonates with the natural frequency of the human body.

5. The electronic device of claim 4, wherein the processor is configured to determine whether the behavior of the vehicle corresponds to a sudden cornering, sudden braking, or sudden acceleration.

6. The electronic device of claim 1, wherein the processor is configured to:
determine whether the motion sickness is caused by the sitting posture of the user; and
correct the sitting posture based on determining that the motion sickness is caused by the sitting posture.

7. The electronic device of claim 6, wherein the processor is configured to:
adjust an amount of air in at least one of (i) a first air tube included in a neck pillow of a vehicle seat of the vehicle or (ii) a second air tube included in a seat cushion of the vehicle seat, or any combination thereof, to thereby adjust load distribution of the sitting posture to be even.

8. The electronic device of claim 6, wherein the processor is configured to:
output a notification suggesting correction of the sitting posture.

9. The electronic device of claim 1, wherein the processor is configured to:
determine whether the motion sickness is caused by a resonance with a natural frequency of a human body; and
perform a resonance avoidance control based on determining that the motion sickness is caused by the resonance with the natural frequency of the human body.

10. The electronic device of claim 9, wherein the processor is configured to:
expand at least one of (i) a first air tube included in a neck pillow of a vehicle seat of the vehicle or (ii) a second air tube included in a seat cushion of the vehicle seat, or any combination thereof, to thereby change the natural frequency of the human body.

11. A control method of an electronic device, the control method comprising:
obtaining, from one or more sensors, motion information including at least one of a sitting posture of a user of a vehicle or driving information of the vehicle, or any combination thereof;
determining whether the user experiences motion sickness based on the motion information; and
providing a motion sickness reduction solution based on determining that the user experiences the motion sickness.

12. The control method of claim 11, wherein the one or more sensors include a pressor sensor and a vehicle sensor, and
wherein obtaining the motion information comprises:
measuring body pressure distribution on a seat back and a seat cushion of a vehicle seat of the vehicle; and
obtaining the driving information of the vehicle using the vehicle sensor.

13. The control method of claim 12, wherein determining whether the user experiences the motion sickness comprises:
determining whether load distribution of the sitting posture is eccentric based on the body pressure distribution; and
determining that the user experiences the motion sickness based on determining that the load distribution of the sitting posture is eccentric.

14. The control method of claim 12, wherein determining whether the user experiences the motion sickness comprises:
determining a behavior of the vehicle based on the driving information of the vehicle;
determining whether an excitation frequency according to the behavior of the vehicle resonates with a natural frequency of a human body; and
determining that the user experiences the motion sickness based on determining that the excitation frequency according to the behavior of the vehicle resonates with the natural frequency of the human body.

15. The control method of claim 14, wherein determining the behavior of the vehicle comprises:
determining whether the behavior of the vehicle is a sudden cornering, sudden braking, or sudden acceleration.

16. The control method of claim 12, wherein providing the motion sickness reduction solution comprises:
determining whether the motion sickness is caused by the sitting posture; and
correcting the sitting posture based on determining that the motion sickness is caused by the sitting posture.

17. The control method of claim 16, wherein correcting the sitting posture comprises:
adjusting an amount of air in at least one of (i) a first air tube included in a neck pillow of the vehicle seat or (ii) a second air tube included in the seat cushion, or any combination thereof, to thereby adjust load distribution in the sitting posture to be even.

18. The control method of claim 16, wherein correcting the sitting posture comprises:
outputting a notification suggesting correction of the sitting posture.

19. The control method of claim 12, wherein providing the motion sickness reduction solution comprises:
determining whether the motion sickness is caused by a resonance with a natural frequency of a human body; and
performing a resonance avoidance control based on determining that the motion sickness is caused by the resonance with the natural frequency of the human body.

20. The control method of claim 19, wherein performing the resonance avoidance control comprises:
expanding at least one of (i) a first air tube included in a neck pillow of the vehicle seat or (ii) a second air tube included in the seat cushion, or any combination thereof, to thereby change the natural frequency of the human body.
